(19) United States
(12) Patent Application Publication
(10) Pub. No.: US 2013/0072888 A1
(43) Pub. Date: Mar. 21, 2013

(54) DISPOSABLE MENSTRUAL PANTY AND METHOD THEREOF

(71) Applicant: Larisa Zorin, Tarzana, CA (US)

(72) Inventor: Larisa Zorin, Tarzana, CA (US)

(21) Appl. No.: 13/236,210

(22) Filed: Sep. 19, 2011

(51) Int. Cl. A61L 15/22; D04H 3/04; A61F 13/72

(52) U.S. Cl. 604/372; 604/396; 28/100

(57) ABSTRACT

A disposable menstrual panty and method thereof. The disposable menstrual panty comprises a front part, a back part, a crotch part positioned between the front part and the back part, a waist portion located at upper peripheral ends of the front part and the back part, a pair of leg openings to receive a person's legs, and an absorbent material attached to the disposable menstrual panty such that the absorbent material is longitudinally disposed on an interior surface of the crotch part and extends rearward in the back part. The disposable menstrual panty is made of a material that is stretchable laterally only in a direction perpendicular to a length of a person's body but not vertically in a direction parallel to the length of a person's body when the disposable menstrual panty is worn by the person.

### BACKGROUND OF THE INVENTION

1. Field of the Invention

The invention herein relates to a disposable menstrual underwear and method relating thereto.

2. Background Information

A disposable underwear is well known in the art. Diapers are, in general, a form of disposable underwear. Also, the brand DEPEND® for adults is an example of disposable underwear. All of these forms of disposable underwear are bulky because such disposable underwear needs to have a larger pad that extends rearward to absorb liquid when a user is in a sitting position. Moreover, the pad would sag downward, which would make it look bulky. Typical fabric used for a disposable underwear in the art does not pull the bulky pad closer to a user's body to make it look less bulky.

### SUMMARY OF THE INVENTION

A disposable menstrual panty of the present invention generally comprises a front part, a back part, a crotch part positioned between the front part and the back part, a waist portion located at upper peripheral ends of the front part and the back part, a pair of leg openings to receive a person's legs, and an absorbent material attached to the disposable menstrual panty such that the absorbent material is longitudinally disposed on an interior surface of the crotch part and extends rearward in the back part. The disposable menstrual panty is made of a material that is stretchable laterally only in a direction perpendicular to a length of a person's body but not vertically in a direction parallel to the length of a person's body when the disposable menstrual panty is worn by the person.

A method of providing a disposable menstrual panty of the present invention generally comprises: providing a fabric made of polypropylene resin; putting an elastic thread through the fabric to bring an elasticity to the fabric such that the fabric becomes stretchable in only one direction; forming a panty from the fabric such that the panty comprises a front part, a back part, a crotch part positioned between the front part and the back part, and a pair of leg openings to receive a person's legs; and attaching an absorbent material to the panty.

### BRIEF DESCRIPTION OF THE DRAWINGS

FIG. 1 is a front view of an embodiment of a disposable menstrual panty of the present invention, showing that the disposable menstrual panty is in its folded position.

FIG. 2 is a front view of the embodiment of the present invention shown in FIG. 1 in its expanded position.

FIG. 3 is a rear view of the embodiment of the present invention shown in FIG. 1 in its expanded position.

FIG. 4 is a side view of the embodiment of the present invention shown in FIG. 1 in its expanded position.

FIG. 5 is a top view of the embodiment of the present invention shown in FIG. 1 in its expanded position.

FIG. 6 is a bottom view of the embodiment of the present invention shown in FIG. 1 in its expanded position.

### DETAILED DESCRIPTION OF THE INVENTION

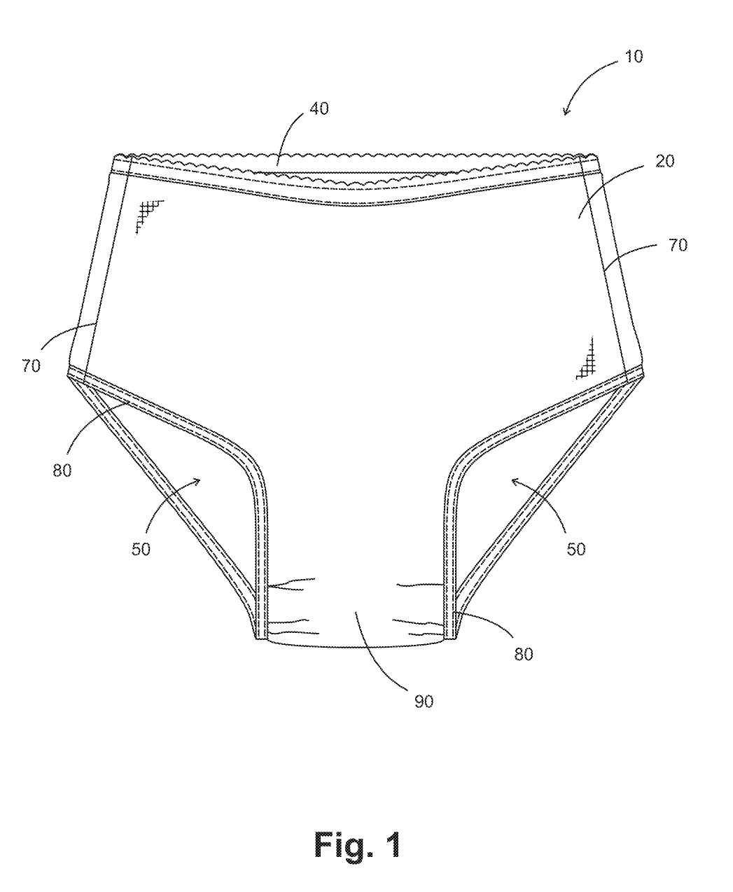

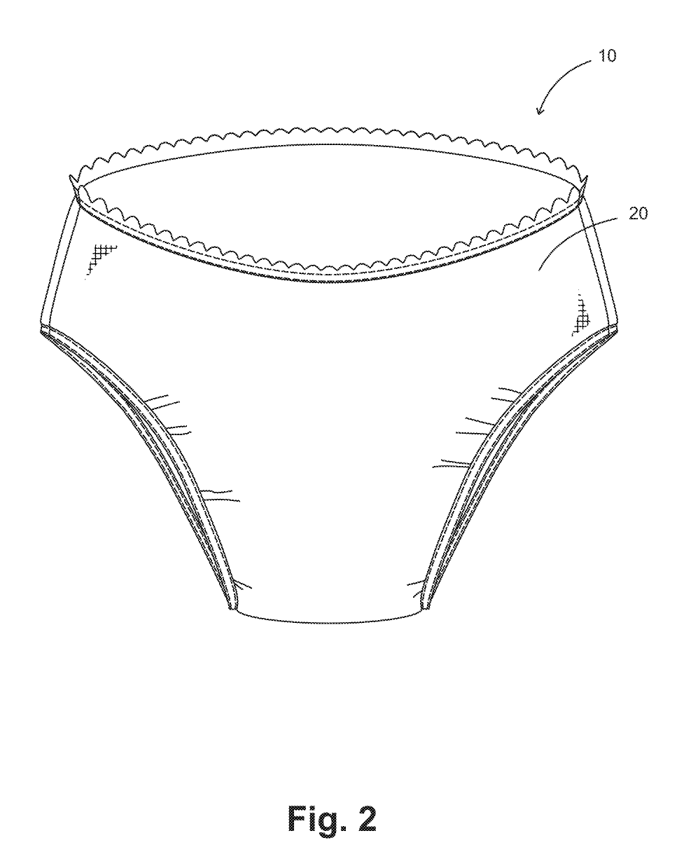

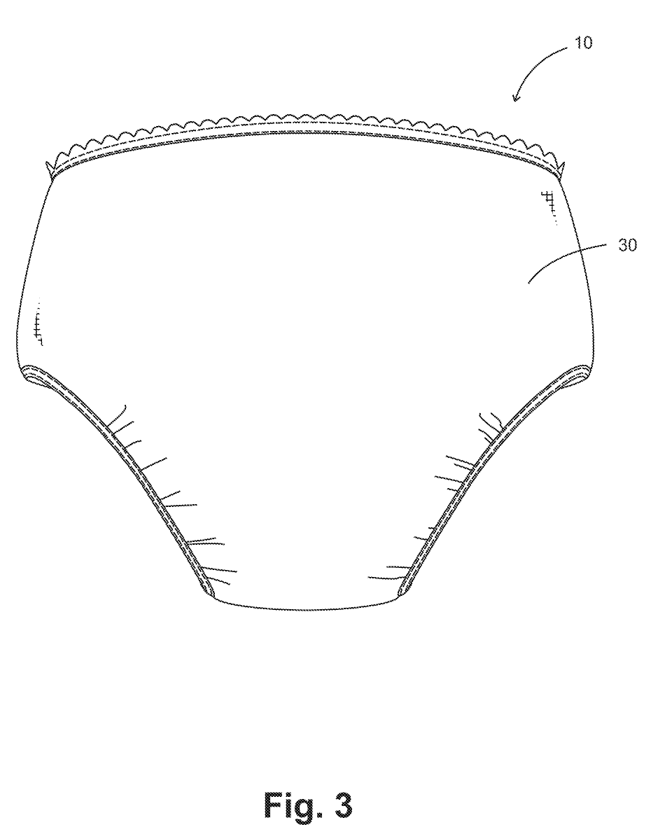

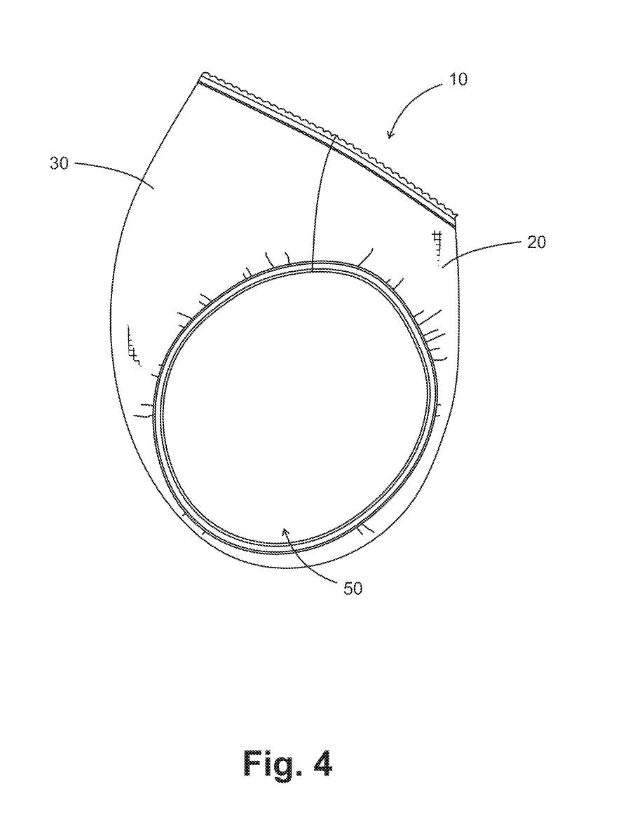

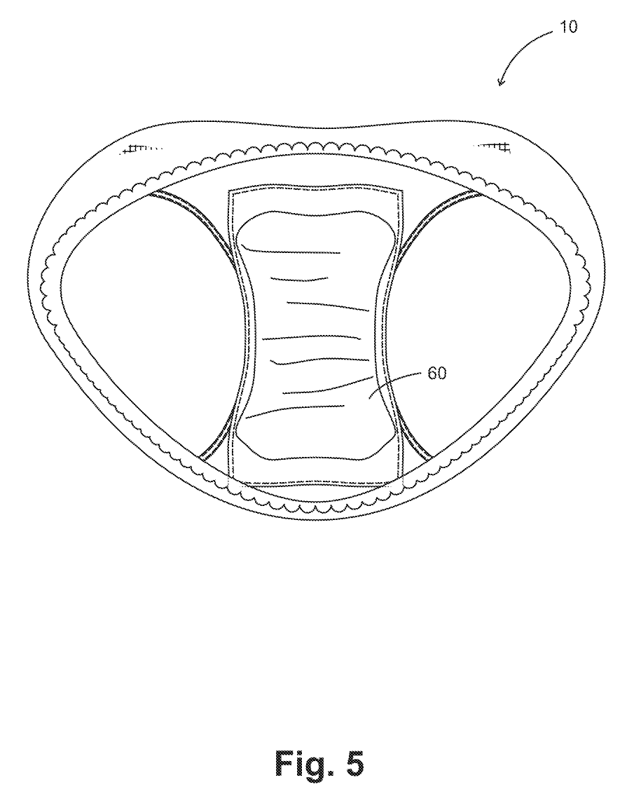

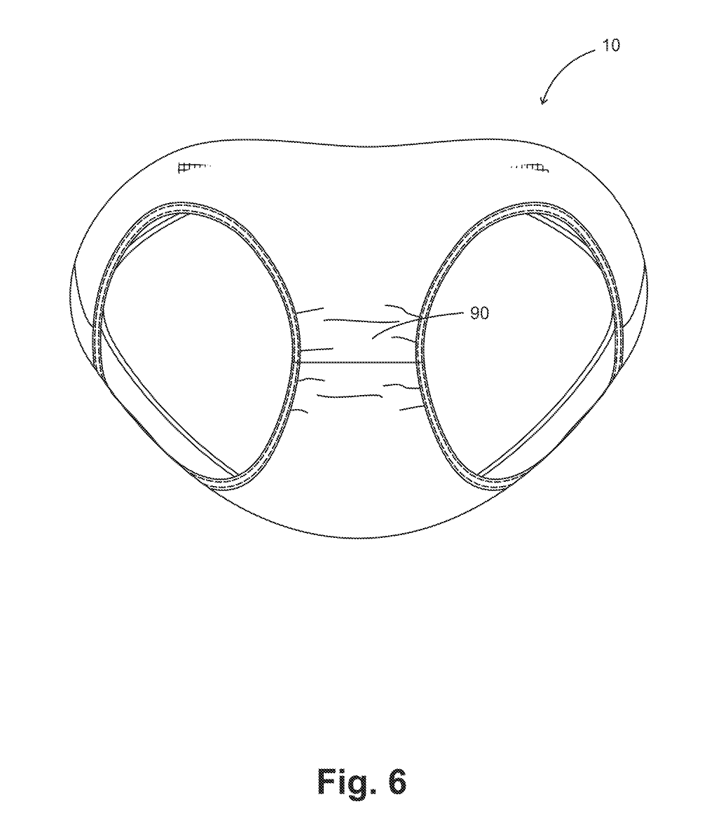

FIGS. 1 through 6 illustrate an embodiment of a disposable menstrual panty of the present invention. A disposable menstrual panty 10 comprises a front part 20, a back part 30, a crotch part 90, a waist portion 40, a pair of leg openings 50 to receive a person's legs, and an absorbent material 60. The crotch part 90 is positioned between the front part 20 and the back part 30. The waist portion 40 is located at upper peripheral ends of the front part 20 and the back part 30. In an embodiment, the waist portion 40 may comprise an elastic band. The absorbent material 60 is attached to the disposable menstrual panty 10 such that the absorbent material 60 is longitudinally disposed on an interior surface of the crotch part 90 and extends rearward in the back part 30. In an embodiment, the absorbent material 60 is an absorbent pad. The absorbent material 60 may be sewn onto the disposable menstrual panty 10 at least along the periphery of the absorbent material 60.

The disposable menstrual panty 10 is made of a material that is stretchable laterally only in a direction perpendicular to a length of a person's body but not vertically in a direction parallel to the length of a person's body when the disposable menstrual panty 10 is worn by the person. In an embodiment, the disposable menstrual panty 10 may be made of an elastic nonwoven material. In another embodiment, the disposable menstrual panty 10 may be made of polypropylene spunbonded nonwovens made from polypropylene resin. The fabric made of polypropylene is water-repellant so that, if there is leakage from the pad, it will not seep through to the user's clothes because the fabric will repel the fluid. It is known that polypropylene resin is imported from Exxon Mobil Corporation. In manufacturing the disposable menstrual panty 10, a fabric may be first made from polypropylene spunbonded nonwovens and an elastic thread may be put through the fabric to bring an elasticity to the fabric such that the fabric becomes stretchable in only one direction.

Since the material used for the disposable menstrual panty of the present invention is stretchable in only one direction, which is perpendicular to a length of a person's body, but not vertically in a direction parallel to the length of a person's body when the disposable menstrual panty 10 is worn by the person, the material allows the panty 10 to stretch laterally to permit it to be put on, then contract around the torso. Accordingly, the material allows formfitting to pull the absorbent pad 60 closer to the person's body to provide a desirable look, which is not bulky. Also, since the material is not stretchable vertically in a direction parallel to the length of a person's body when the disposable menstrual panty 10 is worn by the person, the absorbent pad 60 would not sag downward.

The upper region of the panty 10 is equivalent to a belt holding up the pad region. Because the material of the panty 10 is not stretchable vertically, there should be sufficient coverage above the point where the absorbent pad 60 ends in order to sufficiently keep the pad 60 supported while the panty 10 contracts around the torso. For this reason, a length from a center of the crotch part 90 to a back center of the waist portion 40 in the back part 30 is greater than a length from the center of the crotch part 90 to a front center of the waist portion 40 in the front part 20. Accordingly, the back part 30 of the panty 10 is made much higher than the front part 20 and designed rounded upwards in the waist portion to assure rear coverage.

In the disposable menstrual panty 10, the front part 20 and the back part 30 are sewn together at sides along seams 70 extending from the waist portion 40 to the leg openings 50. The material of the panty 10 may be slightly gathered at the regions near the leg openings 50 to achieve stretch and comfort in the regions. A strip 80 of cloth material may be secured along each of the leg openings 50 to reinforce the nonwoven material therein.

A method of providing a disposable menstrual panty 10 generally comprises: providing a fabric made of polypropylene resin; putting an elastic thread through the fabric to bring an elasticity to the fabric such that the fabric becomes stretchable in only one direction; forming a panty from the fabric such that the panty 10 comprises a front part 20, a back part 30, a crotch part 90 positioned between the front part 20 and the back part 30, and a pair of leg openings 50 to receive a person's legs; and attaching an absorbent material 60 to the panty 10. As discussed above, the fabric is an elastic nonwoven material. In an embodiment, the fabric may be polypropylene spunbonded nonwovens. As described above, the absorbent material 60 is longitudinally disposed on an interior surface of the crotch part 90 and extends rearward in the back part 30. The panty 10 further comprises a waist portion 40 located at upper peripheral ends of the front part 20 and the back part 30. The method of providing a disposable menstrual panty may further comprise sewing the front part 20 and the back part 30 together at sides along seams 70 extending from the waist portion 40 to the leg openings 50. The panty 10 is stretchable laterally only in a direction perpendicular to a length of a person's body but not vertically in a direction parallel to the length of a person's body when the panty 10 is worn by the person. To provide sufficient rear coverage, a length from a center of the crotch part 90 to a back center of the waist portion 40 in the back part 30 is greater than a length from the center of the crotch part 90 to a front center of the waist portion 40 in the front part 20.

While the said detailed description elaborates workable embodiments of the present invention, the said embodiments shall not be construed as a limitation on the patented scope and claims of the present invention and, furthermore, all equivalent adaptations and modifications based on the technological spirit of the present invention shall remain protected within the scope and claims of the invention herein.

What is claimed is:

1. A disposable menstrual panty comprising:
a front part;
a back part;
a crotch part positioned between the front part and the back part;
a waist portion located at upper peripheral ends of the front part and the back part;
a pair of leg openings to receive a person's legs; and
an absorbent material attached to the disposable menstrual panty such that the absorbent material is longitudinally disposed on an interior surface of the crotch part and extends rearward in the back part,
wherein the disposable menstrual panty is made of a material that is stretchable laterally only in a direction perpendicular to a length of a person's body but not vertically in a direction parallel to the length of a person's body when the disposable menstrual panty is worn by the person.

2. The disposable menstrual panty of claim 1, wherein the material is an elastic nonwoven material.

3. The disposable menstrual panty of claim 1, wherein the material is polypropylene spunbonded nonwovens made from polypropylene resin.

4. The disposable menstrual panty of claim 1, wherein the waist portion comprises an elastic band.

5. The disposable menstrual panty of claim 1, wherein a length from a center of the crotch part to a back center of the waist portion in the back part is greater than a length from the center of the crotch part to a front center of the waist portion in the front part.

6. The disposable menstrual panty of claim 1, wherein the front part and the back part are sewn together at sides along seams extending from the waist portion to the leg openings.

7. The disposable menstrual panty of claim 1, wherein the absorbent material is sewn onto the disposable menstrual panty.

8. The disposable menstrual panty of claim 1, wherein the absorbent material is an absorbent pad.

9. A method of providing a disposable menstrual panty, the method comprising:
providing a fabric made of polypropylene resin;
putting an elastic thread through the fabric to bring an elasticity to the fabric such that the fabric becomes stretchable in only one direction;
forming a panty from the fabric such that the panty comprises a front part, a back part, a crotch part positioned between the front part and the back part, and a pair of leg openings to receive a person's legs; and
attaching an absorbent material to the panty.

10. The method of claim 9, wherein the fabric is an elastic nonwoven material.

11. The method of claim 9, wherein the fabric is polypropylene spunbonded nonwovens.

12. The method of claim 9, wherein the absorbent material is longitudinally disposed on an interior surface of the crotch part and extends rearward in the back part.

13. The method of claim 9, wherein the panty further comprises a waist portion located at upper peripheral ends of the front part and the back part.

14. The method of claim 13, wherein a length from a center of the crotch part to a back center of the waist portion in the back part is greater than a length from the center of the crotch part to a front center of the waist portion in the front part.

15. The method of claim 13, which further comprises sewing the front part and the back part together at sides along seams extending from the waist portion to the leg openings.

16. The method of claim 9, wherein the panty is stretchable laterally only in a direction perpendicular to a length of a person's body but not vertically in a direction parallel to the length of a person's body when the panty is worn by the person.
